(19) United States
(12) Patent Application Publication
(10) Pub. No.: US 2020/0271646 A1
(43) Pub. Date: Aug. 27, 2020

(54) COMPOUND CONTAINING THIOESTER GROUP FOR MODIFYING SUBSTRATE SURFACE AND METHOD USING THE SAME

(71) Applicant: National Central University, Taoyuan City (TW)

(72) Inventors: Chen-Han HUANG, Taoyuan City (TW); Wen-Hao CHEN, Taoyuan City (TW)

(21) Appl. No.: 16/796,151

(22) Filed: Feb. 20, 2020

(30) Foreign Application Priority Data
Feb. 21, 2019 (TW) 108105855

(51) Int. Cl. G01N 33/543; C07F 7/18

(52) U.S. Cl. CPC G01N 33/54353; C07F 7/1804

(57) ABSTRACT

A compound containing a thioester group for modifying a substrate surface and a method using the same, the compound containing the thioester group is composed of a compound shown in formula 1:

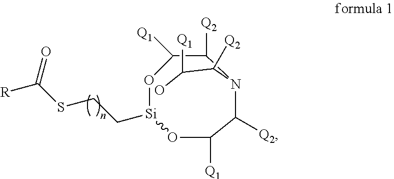

n is an integer of 0 to 30; Q1 and Q2 are each independently a hydrogen atom, a halogen, or an aliphatic hydrocarbon group, an aromatic group, a hydroxyl group, an ether group, an ester group, or an aliphatic hydrocarbon group containing silicon with carbon number of 1 to 6; and R is a hydrogen atom, or an alkyl group, an alkoxy group, or a thiol group containing nitrogen with carbon number of 1 to 6. The method uses water as a medium to modify the substrate surface, and noble metal nanoparticles and/or biomolecules can be immobilized on the modified substrate surface in an aqueous solution with a pH of 5.5 to 7.5.

### FIELD OF THE INVENTION

The present invention relates to a compound containing a thioester group, and more particularly to a compound containing a thioester group for modifying a substrate surface and a method using the same.

### BACKGROUND OF THE INVENTION

In the biomedical sensing technology, the material surface interacts with biomolecules to generate a chemical or physical reaction based on the chemical properties and physical microstructure of the material. In general, silane compounds are often used to modify the surface of the material. Besides, the noble metal nanoparticle is also developed for the use as a signal source for biomolecules identification, wherein the noble metal nanoparticle is immobilized on the material interface to allow the material surface having the ability to specifically identify biomolecules.

Among the methods for immobilizing the noble metal nanoparticles, immobilization reaction could be carried out by attracting amino group-containing silane compounds and noble metals in an electrostatic adsorption manner, for example, ((3-aminopropyl)trimethoxysilane, APTMS) is used for the affinity surface modification of the substrate. However, the amino group is only affinity-immobilized with the metals by the force of electrostatic adsorption, which is weak for the ability of immobilizing the APTMS to the substrate.

Immobilization reaction could also be carried out by covalently bonding noble metals with silane compounds containing mercapto group, for example, ((3-mercaptopropyl)trimethoxysilane, MPTMS) is used for the affinity surface modification of the substrate. However, since the mercapto group is easily deactivated by an oxidation reaction with oxygen in the air, the noble metals cannot be immobilized to the surface of the substrate in pure water or an aqueous solution.

Most of the silanizing self-assembled molecules used for surface modification are water-sensitive, resulting in a decrease in their ability to modify in a wet state. Therefore, traditional silanizing self-assembled molecules must use highly polar organic solvents, but organic solvents often affect the surface of the polymer substrate or destroy its properties. For this reason, the silanizing self-assembled molecules are limited when applied to various substrate materials.

### SUMMARY OF THE INVENTION

In order to solve the above problems, a main object of the present invention is to provide a compound containing a thioester group for modifying a substrate surface, which can effectively inhibit oxidation reaction with oxygen in the air by using an silatrane containing a thioester group, and can be effectively distributed in an aqueous solution by the acting force of hydrogen bond for surface modification of various substrates.

Another main object of the present invention is to provide a method for modifying a substrate surface, which utilizes a carboxyl group as a protecting group and reacts with the thiol end of a silatrane molecule to form a thioester group, and the thioester group can be used to immobilize noble metal nanoparticles or biomolecules to various substrates in an aqueous solution.

In accordance with the above objects, the present invention first provides a compound containing a thioester group for modifying a substrate surface, which is composed of a compound as described in formula 1:

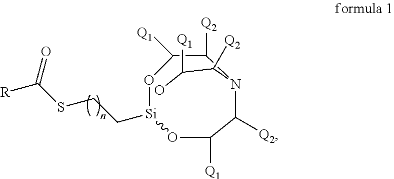

wherein n is an integer of 0 to 30; Q1 and Q2 are each independently a hydrogen atom, a halogen, or an aliphatic hydrocarbon group, an aromatic group, a hydroxyl group, an ether group, an ester group, or an aliphatic hydrocarbon group containing silicon with carbon number of 1 to 6; and R is a hydrogen atom, or an alkyl group, an alkoxy group, or a thiol group containing nitrogen with carbon number of 1 to 6.

The present invention further provides a method of using a compound containing a thioester group, and the compound containing the thioester group is attached to a substrate surface by water as a medium to modify the substrate surface.

The present invention further provides a method for modifying a substrate surface, comprising: preparing a surface modification solution, providing a substrate to be surface-modified, and coating the surface modification solution on a surface of the substrate to be surface-modified to react for completing surface modification of the substrate. Wherein steps of preparing the surface modification solution comprise: providing a (3-mercaptopropyl)trimethoxysilane as a reaction initiator; reacting the reaction initiator with an acid anhydride group to obtain an intermediate product; purifying the intermediate product to obtain a purified intermediate product; adding a triethanolamine to react with the purified intermediate product in a toluene solution to obtain a final reactant; adding a dimethyl sulfoxide to the final reactant to prepare a standard solution; and diluting the standard solution to form the surface modification solution.

In a preferred embodiment of the present invention, after the surface modification of the substrate is completed, the method further comprises a step of immobilizing the surface-modified substrate with noble metal nanoparticles and/or biomolecules in an aqueous solution with a pH of 5.5 to 7.5.

According to the above, the compound containing the thioester group for modifying the substrate surface and the method using the same can be used for surface modification of various substrates such as metals, polymers or glass in an aqueous solution environment, and the noble metal nanoparticles and/or biomolecules are closely attached to the substrate surface through the covalent bond force between the thioester group and the noble metal nanoparticles or biomolecules, allowing the substrate being quickly surface modified and biologically modified, and having the ability to specifically identify biomolecules.

### BRIEF DESCRIPTION OF THE DRAWINGS

FIG. 1A is an atomic force microscope image of Example 1 in accordance with an embodiment of the present invention;

FIG. 1B is an atomic force microscope image of Comparative Example 1 in accordance with an embodiment of the present invention;

FIG. 1C is an atomic force microscope diagram of Comparative Example 2 in accordance with an embodiment of the present invention;

FIG. 2A is an ultraviolet-visible light spectrogram after 30 minutes of the reaction of gold nanoparticles with a glass of Example 3 in accordance with an embodiment of the present invention;

FIG. 2B is a curve diagram of standardized absorbance and reaction time of Example 3 and Comparative Example 3 in accordance with an embodiment of the present invention;

FIG. 2C is a graph of time exposed to air and standardized absorbance of Example 1, Comparative Example 1 and Comparative Example 2 in accordance with an embodiment of the present invention;

FIG. 3A is a curve diagram of standardized absorbance at a wavelength of 520 nm and immobilized time for gold nanoparticles immobilized on a surface of a surface-modified glass in aqueous solutions of different pH in accordance with an embodiment of the present invention;

FIG. 3B is a curve diagram of standardized absorbance at a wavelength of 520 nm and immobilized time for gold nanoparticles immobilized on a surface of surface-modified polymer material in aqueous solutions of different pH in accordance with an embodiment of the present invention; and

FIG. 4 is a curve diagram of surface-enhanced Raman signals of synthetic gold nanoparticles (AuNP-JLR) with specificity for polyglutamine in accordance with an embodiment of the present invention.

### DETAILED DESCRIPTION OF THE PREFERRED EMBODIMENTS

In order to make the above and other objects, features and advantages of the present invention more comprehensible, a compound containing a thioester group for modifying a substrate surface and a method using the same are described below, and related implementations and embodiments thereof are provided to illustrate the present invention and its efficacies specifically.

A main component of the compound containing the thioester group for modifying the substrate surface of the present invention is an silatrane containing a thioester group with a chemical structure as the general formula represented by formula 1:

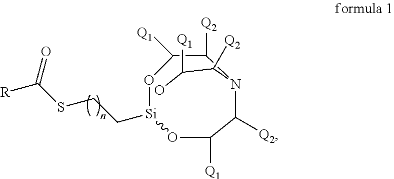

wherein n is an integer of 0 to 30; Q1 and Q2 are independently a hydrogen atom, a halogen, or an aliphatic hydrocarbon group, an aromatic group, a hydroxyl group, an ether group, an ester group, or an aliphatic hydrocarbon group containing silicon with carbon number of 1 to 6; and R is a hydrogen atom, or an alkyl group, an alkoxy group, or a thiol group containing nitrogen with carbon number of 1 to 6.

In the present invention, the silatrane is a tricyclic cage symmetrical structure composed of a silicon nitride bond as a main axis, and has water vapor tolerance and low sensitivity to water, thereby the silatrane is very suitable for surface bio-modification. On the contrary, silane compounds that have been traditionally and widely used in surface coating technology of bio-sensing will cause problems of surface aggregation and unevenness because silane functional groups are easily hydrolyzed.

The compound represented by formula 1 of the present invention contains a thioester group (SCOR), and the double bond oxygen in the thioester group easily undergoes a nucleophilic substitution reaction with water and an acid to form a carboxyl group, which interacts with water molecules to form hydrogen bonds. Thus, the thioester group-containing compound of the present invention is soluble in water. A sol-gel reaction occurs in water to modify the surface of the substrate based on the interaction between the thioester group-containing compound and a hydroxyl group on the substrate, wherein the substrate could be any one of the metals, polymers or glass. Preferably, the thioester group-containing compound of the present invention is bonded to the substrate surface by using water and an aqueous solution such as dichloromethane or ethanol as a medium to modify the substrate surface. Consequently, the present invention is able to reduce the consumption of organic solvents and avoid damage to the substrate surface.

According to the thioester group-containing compound provided by the present invention, the present invention simultaneously discloses a method for modifying a substrate surface, which sequentially comprises steps of (S1) preparing a surface modification solution, and (S2) performing surface modification on the substrate. The above steps are described in detail below.

(S1) Preparing a Surface Modification Solution

The surface modification solution of the present invention contains a silatrane as shown in formula 1, which is a polymer formed by ring-opening polymerization. The reaction formula 1 to the reaction formula 5 shown as following sequentially represent procedure steps for preparing the surface modification solution according to an embodiment of the present invention. First, performing step a as shown in the reaction formula 1: providing a (3-mercaptopropyl)trimethoxysilane represented by formula 2 as a reaction initiator, and reacting the reaction initiator with an acid anhydride group as shown in formula 3 to obtain an intermediate product. Specifically, in an acetonitrile solvent, the (3-mercaptopropyl)trimethoxysilane, which is provided as the reaction initiator, contains a thiol group with nucleophilic sulfur atoms The electron of the nucleophilic sulfur atoms attack the acid anhydride group to perform the ring-opening polymerization. Then, adding pure water as a terminator and terminating the reaction to obtain the intermediate product as shown in formula 4, which is a compound with acid anhydride group, such as acetic anhydride or di-tert-butyl dicarbonate, but is not limited thereto. Further, performing step b as shown in the reaction formula 2: purifying the intermediate product by extraction to obtain a purified intermediate product (as shown in formula 4). Then, performing step c as shown in the reaction formula 3: adding a triethanolamine as shown in formula 5 to react with the purified intermediate product in a toluene solution to obtain a final reactant as shown in formula 6; Then, performing step d as shown in the reaction formula 4: adding a dimethyl sulfoxide to the final reactant to prepare a standard solution, wherein the dimethyl sulfoxide is a polar solvent and is miscible with water to stably store the final reactant; and finally, performing step e as shown in the reaction formula 5: diluting the standard solution with dichloromethane, ethanol or pure water to form the surface modification solution.

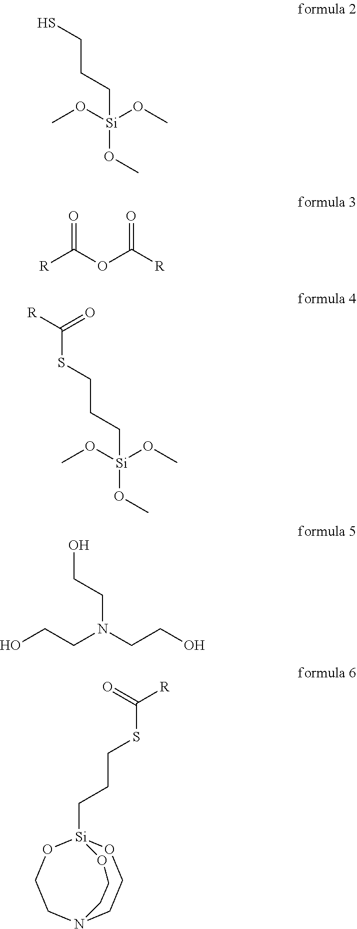

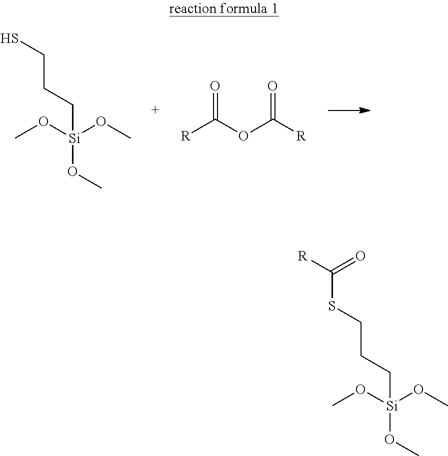

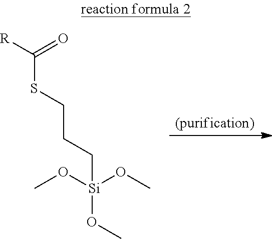

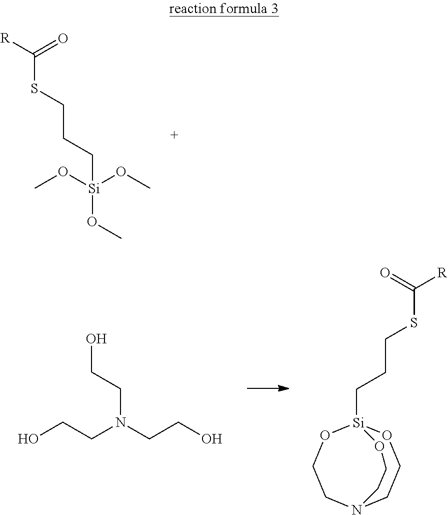

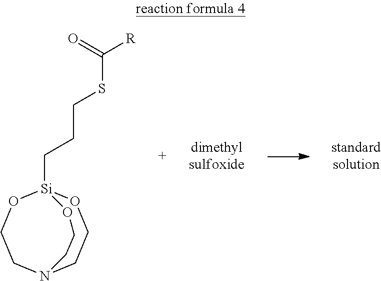

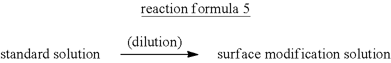

(S2) Performing Surface Modification on the Substrate

In step S2, the substrate to be surface-modified (for example, various substrates such as metals, polymers, or glass, but not limited thereto) is reacted with the surface modification solution. The reaction could be carried out by immersing the substrate in the surface modification solution, or coating the surface modification solution on the substrate surface. The thioester group-containing silatrane which is uniformly dissolved in a solvent, preferably dissolved in pure water, ethanol or an aqueous solution of dichloromethane, is subjected to a sol-gel reaction with a hydroxyl group (—OH) on the substrate. The electrons of the nucleophilic atom (oxygen (O) atom) of the hydroxyl group on the substrate surface attack the silicon (Si) atoms of the thioester group-containing silatrane, causing the Si—O bond to be broken. After the electrons of oxygen atoms attack the silicon (Si) atoms of the thioester group-containing silatrane several times, the thioester group-containing silatrane is bonded to the substrate surface to modify the substrate surface.

In one embodiment, the present invention discloses a method for modifying a substrate surface to immobilize biomolecules on the substrate surface sequentially comprising steps of (S1) preparing a surface modification solution; (S2) performing surface modification on the substrate; and (S3) immobilizing the biomolecules on the substrate surface. Step (S1) and step (S2) are performed as described above, and will not be described again. The following describes the consequent step.

(S3) Immobilizing the Biomolecules on the Substrate Surface

Proteins and nucleotides contained in biomolecules such as antibodies, antigens, enzymes, bacteria, and microorganisms have disulfide bonds. Stability of the disulfide bond is easily destroyed by thiols, causing the position of the disulfide bond being changed. This is called thiol-disulfide exchange reaction. In step S3, the surface-modified substrate is placed in an aqueous solution with a pH of 5.5 to 7.5, and the biomolecules are added into the solution. Accordingly, the thioester group-containing silatrane located on the surface of the surface-modified substrate reacts with an aqueous solution to form a thiol group, and a thiol-disulfide exchange reaction is occurred between the thiol group and the disulfide bonds contained in the biomolecules to immobilize the biomolecules on the substrate surface. For example, ethanol, water, phosphate buffer solution, HEPES buffer solution 2-(4-(2-Hydroxyethyl)piperazin-1-yl)ethanesulfonic acid, acetate buffer solution, etc. are preferred, but the present invention is not limited to the use of only these solvents.

In one embodiment, the present invention discloses a method for modifying a substrate surface to immobilize noble metal nanoparticles on the substrate surface. The method sequentially comprises steps of (S1) preparing a surface modification solution; (S2) performing surface modification on the substrate; and (S3) immobilizing the noble metal nanoparticles on the substrate surface. Step (S1) and step (S2) are performed as described above, and will not be described again. The following describes the consequent step.

(S3) Immobilizing the Noble Metal Nanoparticles on the Substrate Surface

The thioester group-containing silatrane located on the surface of the surface-modified substrate has the thioester group to undergo a nucleophilic substitution reaction with water and an acid, and the thioester group reacts with an aqueous solution with a pH of 5.5 to 7.5 to form a thiol group, thereby the thiol group is covalently bonded with the noble metal nanoparticles to immobilize the noble metal nanoparticles to the surface of the surface-modified substrate. For example, ethanol, water, phosphate buffer solution, HEPES buffer solution 2-(4-(2-Hydroxyethyl)piperazin-1-yl)ethanesulfonic acid, acetate buffer solution, etc., are preferably used as a reaction environment for immobilization of the noble metal nanoparticles, but the present invention is not limited to the use of only these solvents. The noble metal nanoparticles immobilized on the substrate is used to identify targets such as proteins, nucleic acids, small molecules and the like, and is used as a signal source for detecting the target. In addition, due to the magnetic, electrochemical and optical properties of the noble metal nanoparticles, they can also be applied to in vitro detection. Furthermore, after the step (S3), (S4) is performed to bond the biomolecules to the noble metal nanoparticles. Since the noble metal nanoparticles are covalently bonded to the surface of the surface-modified substrate by the thiol group, the disulfide bonds contained in the biomolecules undergo a thiol-disulfide exchange reaction with the thiol group, and the biomolecules are bonded with the noble metal nanoparticles.

The details of the various different examples of the present invention are provided below to more clearly illustrate the present invention, but the present invention is not limited to the following examples.

### Ability Test of Substrate Surface Modification

### Example 1

(3-mercaptopropyl)trimethoxysilane and di-tert-butyl dicarbonate are placed in a beaker containing an acetonitrile solvent to react for 12 hours to obtain an intermediate product; then, the intermediate product is purified by extraction at room temperature; the purified intermediate product and triethanolamine are placed in a toluene solution to react for 6 hours to obtain a tert-butoxycarbonyl thioester alkylated silatrane; dimethyl sulfoxide is added to prepare a standard solution with a concentration of 1M, and the standard solution is stored; the standard solution is diluted 1,000 times in ethanol to prepare a surface modification solution A with a final concentration of 1 mM; and the surface modification solution A is dip-coated on a glass to react for 30 minutes, then the glass is rinsed with deionized water and alcohol, and dried by cold air to obtain a surface-modified glass.

### Example 2

The surface modification solution A is prepared in the same manner as in Example 1, and the surface modification solution A is dip-coated on a polymer material to react for 30 minutes, and then the polymer material is rinsed with deionized water and alcohol, and dried by cold air to obtain a surface modified polymer material.

### Comparative Example 1

(3-mercaptopropyl)trimethoxysilane is prepared into a solution with a concentration of 1M using dimethyl sulfoxide, and the 1M solution is diluted with ethanol into a surface modification solution B with a final concentration of 1 mM; the surface modification solution B is dip-coated on a glass to react for 30 minutes, and then the glass is rinsed with deionized water and alcohol, and dried by cold air to obtain a surface-modified glass.

### Comparative Example 2

A surface-modified glass is obtained in the same manner as in the preparation method of Comparative Example 1, except that (3-mercaptopropyl)trimethoxysilane is substituted with the (3-mercaptopropyl)silatrane.

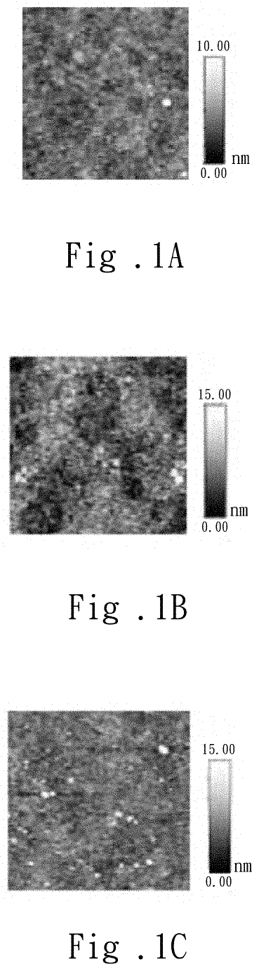

The cross-sectional views of Example 1, Comparative Example 1, and Comparative Example 2 are observed by an atomic force microscope. The results are shown in FIG. 1A, FIG. 1B, and FIG. 1C. FIG. 1A is an atomic force microscope diagram of Example 1 in accordance with an embodiment of the present invention, and it can be known from FIG. 1A that the surface roughness (RMS) is 350 pin. FIG. 1B is an atomic force microscope diagram of Comparative Example 1 in accordance with an embodiment of the present invention, and it can be known from FIG. 1B that the surface roughness (RMS) is 450 pin; and FIG. 1C is an atomic force microscope diagram of Comparative Example 2 in accordance with an embodiment of the present invention, and it can be known from FIG. 1C that the surface roughness (RMS) is 380 pin. As a result, the surface roughness is optimized in Example 1, and it indicates that the modified-surface of the glass treated with the tert-butoxycarbonyl thioester alkylated silatrane is more even than Comparative Examples 1 and 2. This result shows that the thioester group has a better ability to modify the substrate surface than the thiol group.

### Immobilization Test of Noble Metal Nanoparticles

### Example 3

The surface-modified glass of Example 1 is placed in an ethanol solution to react with gold nanoparticles. The absorbance at a wavelength of 520 nm is measured by an ultraviolet-visible light spectrometer after different reaction times; and after 30 minutes of reaction, the absorbances at various wavelengths are measured by an ultraviolet-visible light spectrometer.

### Comparative Example 3

The surface-modified glass of Comparative Example 2 is placed in an ethanol solution to react with the gold nanoparticles, and the absorbance at a wavelength of 520 nm is measured by an ultraviolet-visible light spectrometer after different reaction times.

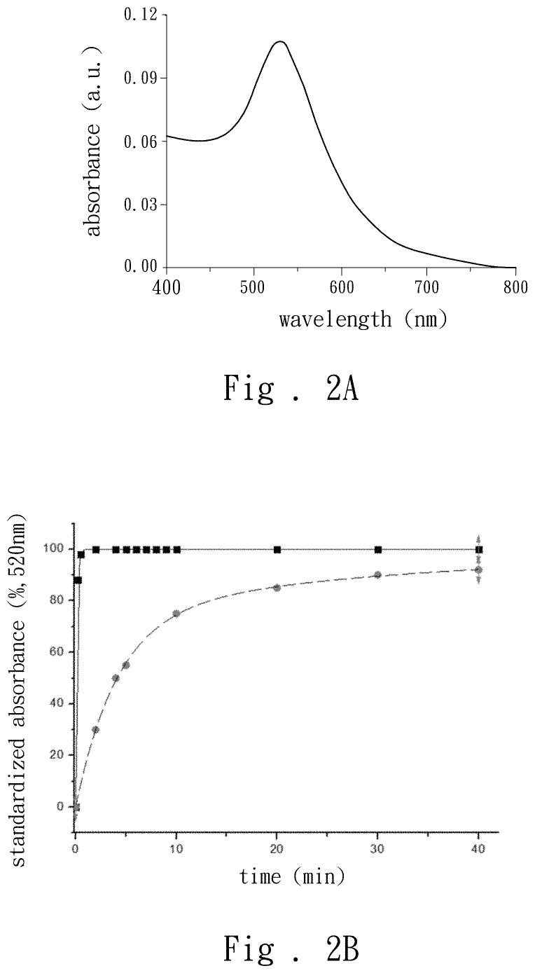

The results are shown in FIG. 2A. FIG. 2A is an ultraviolet-visible light spectrogram after 30 minutes of the reaction of gold nanoparticles with a glass of Example 3 in accordance with an embodiment of the present invention. When the gold nanoparticles are irradiated with light with a wavelength of 500 nm to 550 nm, the strongest absorbance (a.u.) is obtained. This optical property is verified by FIG. 2A, demonstrating that the gold nanoparticles of Example 3 can be immobilized on a glass that is surface-modified with the tert-butoxycarbonyl thioester alkylated silatrane in an ethanol solution.

In accordance with an embodiment of the present invention, the standardized absorbance of Example 3 and Comparative example 3 solutions are measured and plotted against the time of the reaction to obtain FIG. 2B. As shown in FIG. 2B, Example 3 is indicated by ▪, and Comparative Example 3 is indicated by ●. The standardized absorbance of the gold nanoparticles of Example 3 reaches 100% in a short time, while the reaction time required for the standardized absorbance of the gold nanoparticles of Comparative Example 3 to reach 100% exceeds 30 minutes. From this, it is understood that the surface modification with the tert-butoxycarbonyl thioester alkylated silatrane is more efficient for immobilization of the noble metal nanoparticles than the surface modification with the (3-mercaptopropyl)silatrane.

Storage Stability Test

The surface-modified glasses of Example 1, Comparative Example 1, and Comparative Example 2 are exposed to air for several hours, and then the surface-modified glasses are placed in an ethanol solution to react with the gold nanoparticles for 30 minutes, and the absorbances at a wavelength of 520 nm are measured by an ultraviolet-visible light spectrometer. A plurality of the surface-modified glasses of Example 1, Comparative Example 1, and Comparative Example 2 are respectively exposed to air for different reaction times. All the absorbance data measured after reacting with the gold nanoparticles are converted into the standardized absorbances, and are corresponded to the exposure times to be plotted in FIG. 2C. FIG. 2C is a graph of time exposed to air and standardized absorbance of Example 1, Comparative Example 1 and Comparative Example 2 in accordance with an embodiment of the present invention. As shown in FIG. 2C, the surface modification with the (3-mercaptopropyl)trimethoxysilane in Comparative Example 2 is inferior in the ability to immobilize the gold nanoparticles. After exposure to air for 24 hours, it is impossible to immobilize the gold nanoparticles on the glass. For the surface modification with the (3-mercaptopropyl)silatrane in Comparative Example 2, the gold nanoparticles can be immobilized on the glass. But after being exposed to air for a long period of time, immobilization is performed, and in terms of exposure time of 12 hours, the immobilization effect of the gold nanoparticles is significantly inferior to that of Example 1. It is confirmed that oxidation reaction is not easy to happen in Example 1 in which the surface modification is treated with the tert-butoxycarbonyl thioester alkylated silatrane because of containing of the thioester group, and it is able to withstand exposure to air for a long period of time without affecting the immobilization ability of the gold nanoparticles.

pH Test of the Reaction Environment

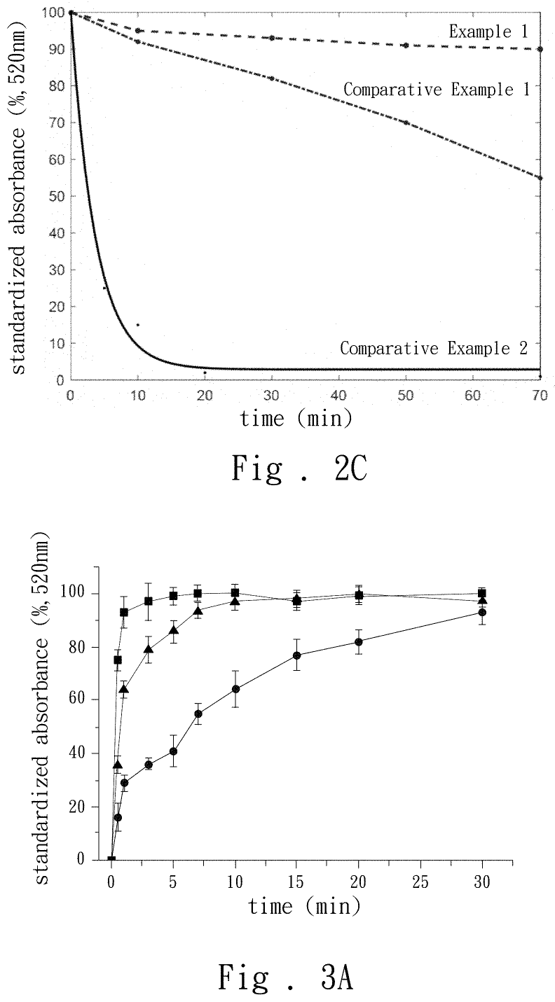

Three sheets of the surface-modified glass of Example 1 are placed in a phosphate buffer solution with a pH of 5 and 9, as well as pure water respectively, and reacted with the gold nanoparticles. The absorbances at a wavelength of 520 nm are measured by an ultraviolet-visible light spectrometer after different reaction times. The standardized absorbances of three sets of the gold nanoparticles corresponding to an aqueous solution with a pH of 5, 7, and 9 at a wavelength of 520 nm are plotted as shown in FIG. 3A, the reaction in pH 5 is indicated by ▪; the reaction in pure water is indicated by ▴; and the reaction in pH 9 is indicated by ●.

The surface-modified polymer material sheets of Example 2 are placed in a phosphate buffer solution with a pH of 5 and 9, as well as pure water respectively, and reacted with the gold nanoparticles. The absorbances at a wavelength of 520 nm are measured by an ultraviolet-visible light spectrometer after different reaction times. The standardized absorbances of three sets of the gold nanoparticles corresponding to an aqueous solution with a pH of 5, 7, and 9 at a wavelength of 520 nm are plotted as shown in FIG. 3B, the reaction in pH 5 is indicated by ▪; the reaction in pure water is indicated by ▴; and the reaction in pH 9 is indicated by ●.

FIG. 3A is a graph of standardized absorbance at a wavelength of 520 nm and immobilized time for gold nanoparticles immobilized on a surface of a surface-modified glass in aqueous solutions of different pH in accordance with an embodiment of the present invention; and FIG. 3B is a graph of standardized absorbance at a wavelength of 520 nm and immobilized time for gold nanoparticles immobilized on a surface of a surface-modified polymer material in aqueous solutions of different pH in accordance with an embodiment of the present invention. It can be known from FIGS. 3A and 3B that the gold nanoparticles have poor ability in immobilizing to the surface-modified glass or polymer material in an alkali aqueous solution. It is preferred to immobilize on the surface of the surface-modified glass or the surface of the surface-modified polymer material in a slightly acidic or neutral aqueous solution.

### Identification Ability Test of Biological Specificity

### Example 4

A 96-well plastic plate being surface modified with a tert-butoxycarbonyl thioester alkylated silatrane is prepared in the same manner as in Example 1, and the gold nanoparticles and the modified 96-well plastic plate are reacted in an ethanol solution for immobilized reaction to form sensing points. 5 μL of a peptide sequence solution is added to each of the sensing points to form a biosensing wafer. This peptide sequence has a specific ability to identify the synthesized polyglutamine but does not contain tryptophan. Polyglutamine is added to the biosensing wafer, and after rinsing with deionized water, synthetic gold nanoparticles (AuNP-JLR) with specific identification ability for polyglutamine are added. The added polyglutamine is immobilized to a surface of the wafer by specifically bonding to the peptide sequence on the surface of the biosensing wafer, and the gold nanoparticles (AuNP-JLR) are also immobilized on the surface of the wafer. The gold nanoparticles on the surface of the wafer are close to the gold nanoparticles (AuNP-JLR) due to the polyglutamine, the tryptophan on the gold nanoparticles (AuNP-JLR) can be used as a signal source for sandwich detection, and a surface-enhanced Raman spectrometer is used to detect the signal strength.

### Comparative Example 4

A biosensing wafer is prepared in the same manner as in Example 4, a protein A is added to the biosensing wafer, and after rinsing with deionized water, synthetic gold nanoparticles (AuNP-JLR) with specific identification ability for polyglutamine are added, and a surface-enhanced Raman spectrometer is used to detect the signal strength.

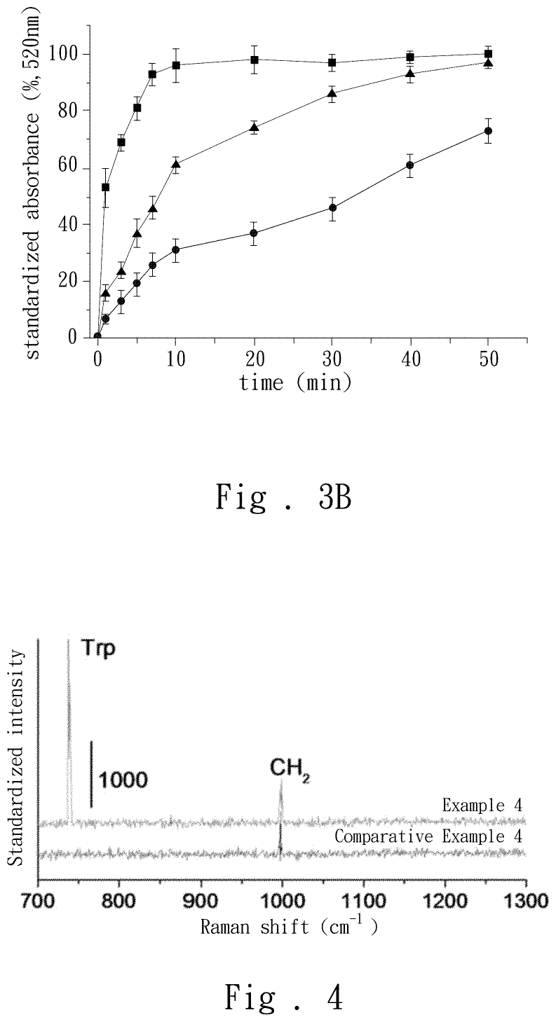

The results are shown in FIG. 4. FIG. 4 is a graph of surface-enhanced Raman signals of synthetic gold nanoparticles (AuNP-JLR) with specificity for polyglutamine in accordance with an embodiment of the present invention. The Raman signal of Example 4 has a significant change, while the Raman signal of Comparative Example 4 is less fluctuating, confirming that the biosensing wafer only produces surface-enhanced Raman spectrum effects on polyglutamine, but does not work on the proteins A, and has the identification ability of biospecificity.

In summary, the thioester group-containing compound of the present invention can modify the substrate surface by using water as a medium, without causing contamination of the substrate with organic solvents, and utilize the protective effect of the thioester group on the thiol end to allow the surface-modified substrate to be stably stored in air. The method for modifying the substrate surface of the present invention is selectively most effective in immobilizing the noble metal nanoparticles and/or biomolecules on the surface of the surface-modified substrate in an aqueous solution with a pH of 5.5 to 7.5, and has the identification ability of biospecificity. Therefore, it can facilitate the research and testing of the noble metal nanoparticles in the field of biomedical technology, for example, clinical applications such as in vitro detection, in vivo drug delivery, target treatment, gene therapy.

What is claimed is:

1. A compound containing a thioester group for modifying a substrate surface, comprising a compound as described in formula 1: 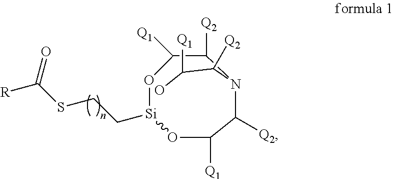
wherein n is an integer of 0 to 30; Q1 and Q2 are each independently a hydrogen atom, a halogen, or an aliphatic hydrocarbon group, an aromatic group, a hydroxyl group, an ether group, an ester group, or an aliphatic hydrocarbon group containing silicon with carbon number of 1 to 6; and R is a hydrogen atom, or an alkyl group, an alkoxy group, or a thiol group containing nitrogen with carbon number of 1 to 6.

2. The compound containing the thioester group as claimed in claim 1, wherein the compound and a noble metal nanoparticle are covalently bonded to a surface of a substrate.

3. The compound containing the thioester group as claimed in claim 2, wherein the substrate is a metal, a polymer or a glass.

4. A method of using the compound containing the thioester group as claimed in claim 1, wherein the compound is attached to a surface of a substrate by water as a medium to modify the surface of the substrate.

5. The method of using the compound containing the thioester group as claimed in claim 4, wherein the substrate is a metal, a polymer or a glass.

6. A method for modifying a substrate surface, comprising:
preparing a surface modification solution, comprising:
providing a (3-mercaptopropyl)trimethoxysilane as a reaction initiator;
reacting the reaction initiator with an acid anhydride group to obtain an intermediate product;
purifying the intermediate product to obtain a purified intermediate product;
adding a triethanolamine to react with the purified intermediate product in a toluene solution to obtain a final reactant;
adding a dimethyl sulfoxide to the final reactant to prepare a standard solution; and
diluting the standard solution to form the surface modification solution;
providing a substrate to be surface-modified; and
coating the surface modification solution on a surface of the substrate to be surface-modified to react for completing surface modification of the substrate.

7. The method for modifying the substrate surface as claimed in claim 6, wherein the step of diluting the standard solution comprises using dichloromethane, ethanol or pure water.

8. The method for modifying the substrate surface as claimed in claim 6, wherein the substrate is a metal, a polymer or a glass.

9. The method for modifying the substrate surface as claimed in claim 6, wherein the step of coating the surface modification solution on the surface of the substrate to be surface-modified to react comprises a sol-gel reaction.

10. The method for modifying the substrate surface as claimed in claim 6, wherein after completing surface modification of the substrate, the method further comprises a step of immobilizing the surface-modified substrate with a noble metal nanoparticle and/or a biomolecule in an aqueous solution with a pH of 5.5 to 7.5.
